(19) United States
(12) Patent Application Publication
(10) Pub. No.: US 2005/0282266 A1
(43) Pub. Date: Dec. 22, 2005

(54) Polymerase chain reactor

(75) Inventors: Chun-Nan Teng, Kaohsung City (TW); Terry B.J. Kuo, Ji-An Township (TW)

Correspondence Address: DENNISON, SCHULTZ, DOUGHERTY & MACDONALD, SUITE 105, 1727 KING STREET, ALEXANDRIA, VA, 22314-2700, US

(73) Assignees: Chun-Nan Teng; Terry B.J. Kuo; WE GENE TECHNOLOGIES INC.

(21) Appl. No.: 10/871,037

(22) Filed: Jun. 21, 2004

(51) Int. Cl. C12M 1/38

(52) U.S. Cl. 435/286.1; 435/303.1

(57) ABSTRACT

A polymerase chain reactor, comprises a temperature measuring system, a temperature controlling system and a human machine operational interface, whereby, the temperature in the course of the polymerase chain reaction detected by a temperature sensor disposed in a reaction tube is converted into an electric signal via analog/digital conversion, this electric signal is transmitted into the microcomputer where it is transformed via an electric signal/temperature transformation program and is then displayed on the operational interface; such that, as the user sets the temperature program for the polymerase chain reaction, the microcomputer can control and adjust based on the reaction temperature program set by the user; and said human machine operational interface can command the microcomputer to output and display a signal such that the operator can select an automatic administration procedure or set condition based on the operation procedure.

### BACKGROUN OF THE INVENTION

1. Field of the Invention

The invention relates to a polymerase chain reactor characterized in that it can amplify accurately a particular nucleotide sequence within a short time, and finds applications in fields of clinical medicine, diagnosis of hereditary disease, detection of virus infection, improvement in agriculture, examination of food, forensic identification of crime, environmental science and molecular revolution.

2. Description of the Prior Art

Polymerase chain reaction (PCR) is a new technique developed by Dr. Mullis of Cetus Company, USA in 1983, who won Nobel Prize in 1993 due to this technique. The development of this technique caused a great impact, including, for example, the whole biological medicine.

Since the development of the PCR technique, a milestone for molecular biology and biological technology had been set up, and relative publications and applications had been more than can be listed. Briefly, PCR is a reaction process in a thermal cycle comprising cyclic reactions among three temperature points. These temperature points includes a high temperature point of about 95° C., a low temperature point of about 52-55° C., and a medium temperature point of about 72° C., as illustrated in FIG. 1. The time length required for the reaction at each temperature point may be different, for example, the time required at the medium temperature may be longer, while that at the high temperature point may be shorter.

At the high temperature point, the object is to separate double strand DNA in to single strand DNA. Then, at low temperature point, an exogenous DNA fragment, i.e., a primer will be annealed to a proper position on the two DNA strand and act as the leader during the synthesis of the DNA. Whereas, at the medium temperature point, a Tag polymerase will be annealed on the DNA by using the primer as the target, as illustrated in FIG. 2.

After addition of suitable bases (dNTPs), a synthesis and extending reaction along the DNA molecule will be proceeded using corresponding bases in a mode of pairing as A=T and G=C. Once a denaturation-annealing-extension cycle being complete, the amount of the DNA fragment becomes double. While n cycles have been carried out, the DNA fragments can be amplified greatly into 2ₙ-fold (as illustrated in FIG. 3). Consequently, in spite of relatively small amount of a genetic substance, by means of PCR amplification, sequencing of a gene, detection of a disease, expression of a gene and the like can be facilitated dramatically.

Referring to the clinical medicine journal in recent years, more and more evidences indicated that the drug resistance developed by bacteria has become a serious problem. An obvious example had been the pulmonary tuberculosis. Because of the generation of drug-resistant strain of tubercle bacillus, the epidemic status of the pulmonary tuberculosis becomes a potential concern. Its main cause resides on the massive abuse of the antibiotics that leads the development of resistance to antibiotics by many bacteria. In view of billion US dollars and decade needed for the research and development of an antibiotic, the number of usable antibiotics for human being will be inevitably less as the drug resistance of bacteria becomes stronger. Accordingly, a rapid molecular diagnostic result can provide a physician a specific type and amount of antibiotic. However, this rapid PCR diagnostic technique can be carried only in a large hospital or medical center, in spite of the high popularity of the modern medical therapy.

A general clinic can use rarely a medical diagnostic equipment of the kind for the following two reasons:
1. The price of this equipment is from about 3,000 to about 10,000 US dollars, a relatively high price for a general clinic; and
2. Most of the PCR equipment are more suitably used for large-scale examination such as, for example, in a big hospital, a medical center or a research unit, and are not appropriate for a general clinic or a populace.

In view of the foregoing disadvantages associated with the conventional structure, the inventor had devoted to improve it and after an intensive study, has provided a polymerase chain reactor of an low price, easy operation and rapid diagnosis, which is useful for various hospitals and general clinics, academic units or popular consumers, and hence accomplished the invention.

### SUMMARY OF THE INVENTION

Accordingly, one object of the invention is to provide a polymerase chain reactor for rapid diagnosis.

Another object of the invention is to provide a polymerase chain reactor of low price and easy operation.

To achieve the above-described objects, polymerase chain reactor according to the invention comprises temperature measurement, temperature control and a man machine interface characterized in that a temperature sensor is disposed in the reaction tube to convert the temperature in the course of the reaction into an electronic signal by means of a analog/digital converter; the signal is then transmitted into a microcomputer where a transforming process of electronic signal into temperature is carried out and the temperature thus obtained in displayed on the operation interface. When the user has set the procedure of the polymerase chain reaction, the microcomputer will control and adjust the process based on the reaction temperature schedule thus determined and said human machine interface will command the microcomputer to output and display the signal such that the operator can select an automatic process or set conditions in accordance with the operation procedure.

The novelty and other features of the invention will be apparent from the following detailed description in conjunction with the accompanied drawings.

### BRIEF DESCRIPTION OF THE DRAWINGS

The invention, as well as its many advantages, may be further understood by the following detailed description and drawings in which:

FIG. 1 is a schematic view of the PCR reaction process according to the invention;

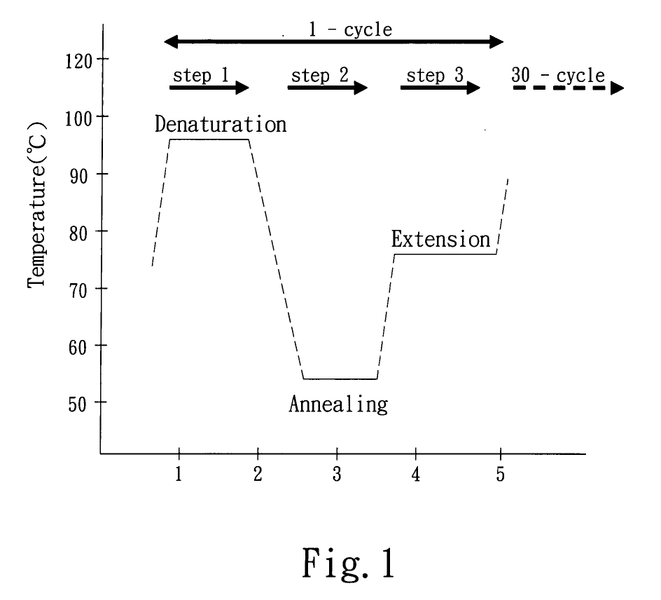

FIG. 2 is a schematic view showing the relationship between the amplification of a genetic molecule and the temperature during a PCR process;

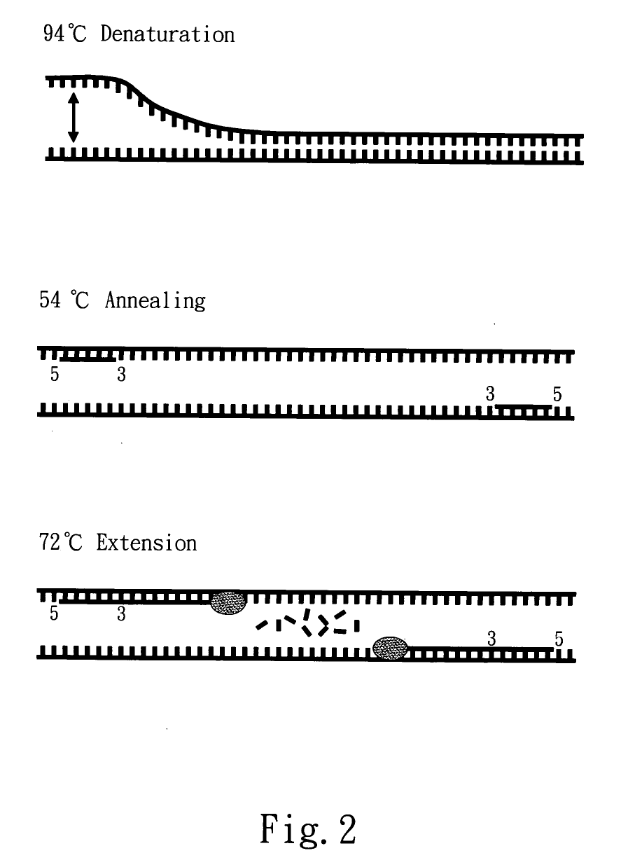

FIG. 3 is a schematic view showing the process of the amplification of a genetic molecule during a PCR process;

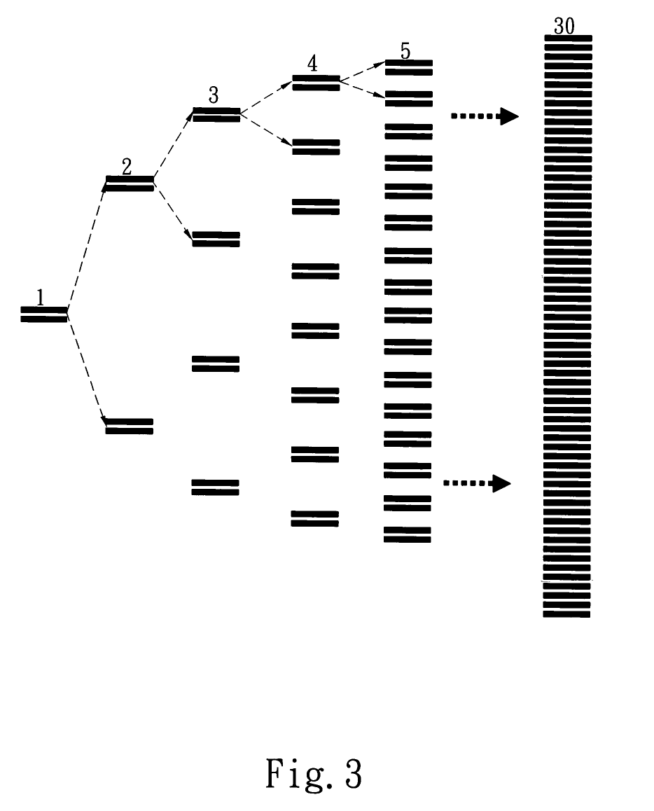

FIG. 4 is the block flowchart diagram illustrating the polymerase chain reactor according to the invention;

FIG. 5 is the control circuit for temperature detection according to the invention;

FIG. 6 is the circuit for voltage-current transformation and the switch;

FIG. 7 is the circuit for controlling the fans according to the invention;

FIG. 8 is the schematic view showing the position of the fans;

FIG. 9 it the schematic view showing the position of the temperature sensor;

FIG. 10 is the planar schematic view of the PCR tank according to the invention;

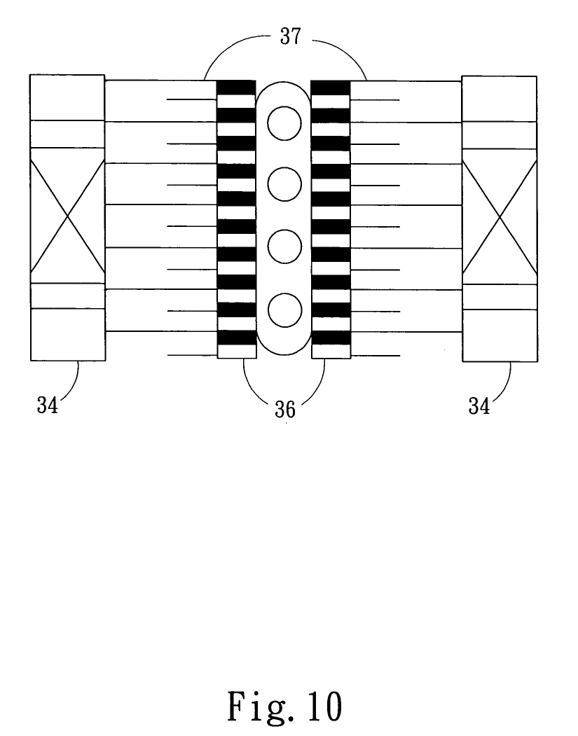

FIG. 11 is photographs showing electrophoresis results of the PCR test on E. Coli tRNA₂^{fMet}, wherein the line 5 and 6 in the photograph shows the electrophoresis result obtained according to the invention, while the line 2 and 3 in the photograph shows the result obtained using Primus 25 legal PCR;

FIG. 12 is the temperature curve displayed on the output interface after carrying out 30 thermal cycles, wherein the PCR amplification tacks place at temperature among 95° C., 55° C., and 72° C.

FIG. 13 shows a comparison between a US PCR machine and a German PCR machine; and

FIG. 14 shows a comparison between PCR machine according to the invention and a conventional PCR machine.

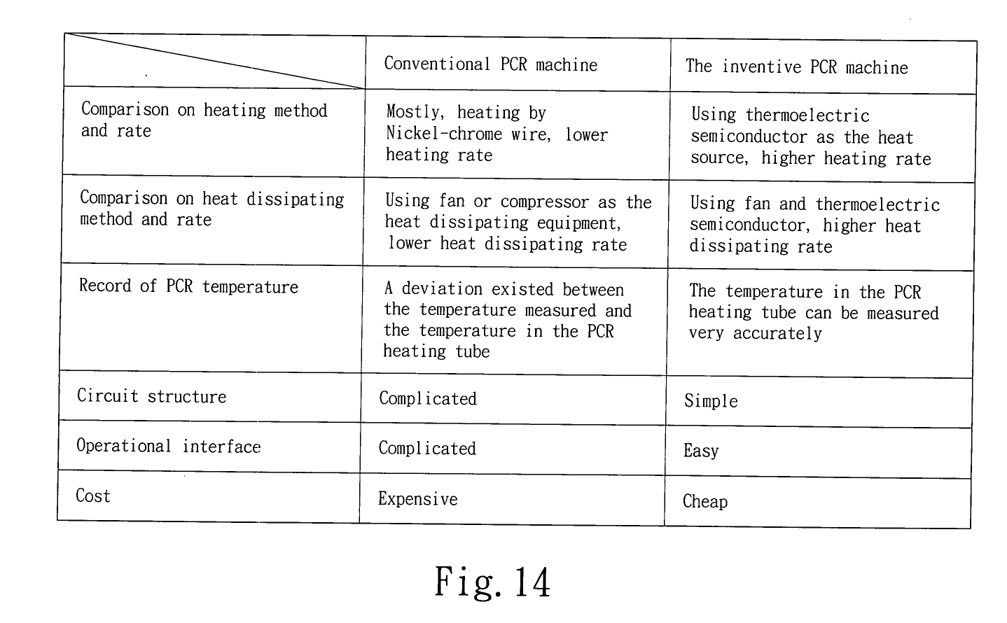

### DETAILED DESCRIPTION OF THE PREFERRED EMBODIMENTS

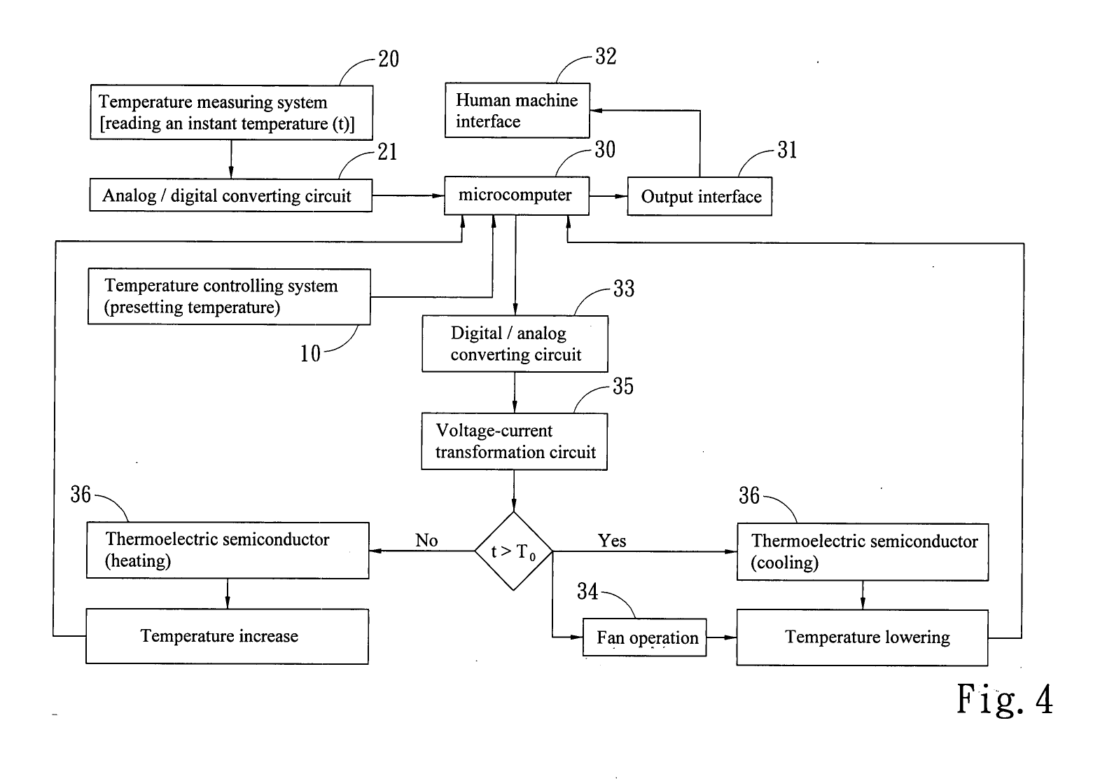

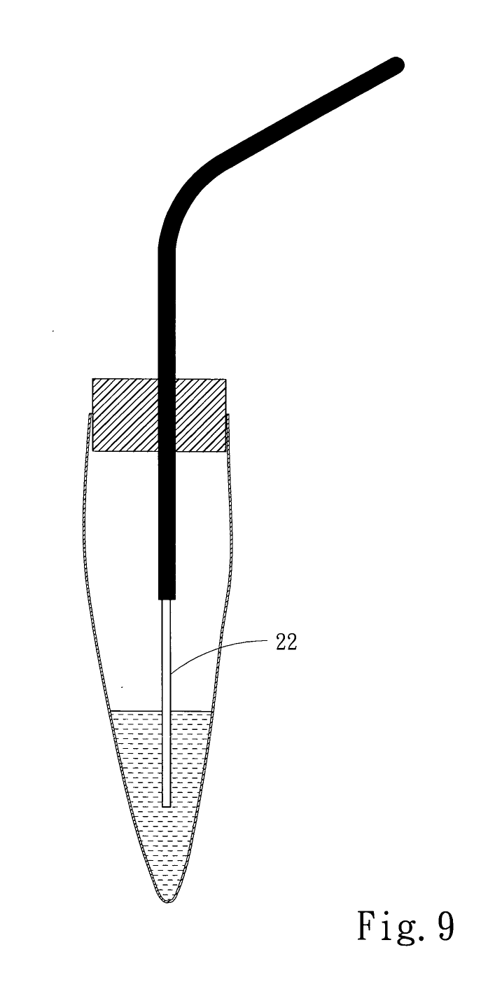

Referring to FIG. 4, the polymerase chain reactor according to the invention comprises essentially of a temperature controlling system 10, a temperature sensing system 20 and a human machine interface 32. Said temperature controlling system 10 can be used by the user to set temperature. Said temperature-sensing system 20 can function as follow: a Negative Temperature Thermistor 22 is placed in a PCR tube (FIG. 9), where temperature of the liquid in the PCR tube might influence the electric resistance of the thermistor 22, and upon applying an external standard voltage, the thermistor will produce a response as an elevated voltage or a reduced voltage. The signal thus produced will be transferred to the analog/digital converter 21 where signal sampling can be carried out 10 times every second. At the same time, signal analysis is performed in a microcomputer 30, and after operational treatment by the microcomputer 30, this signal will be converted into temperature display mode.

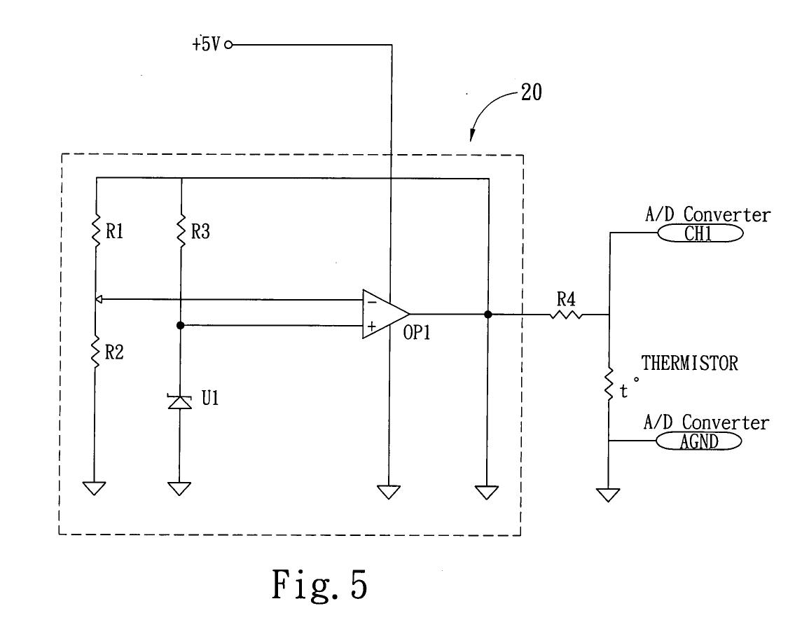

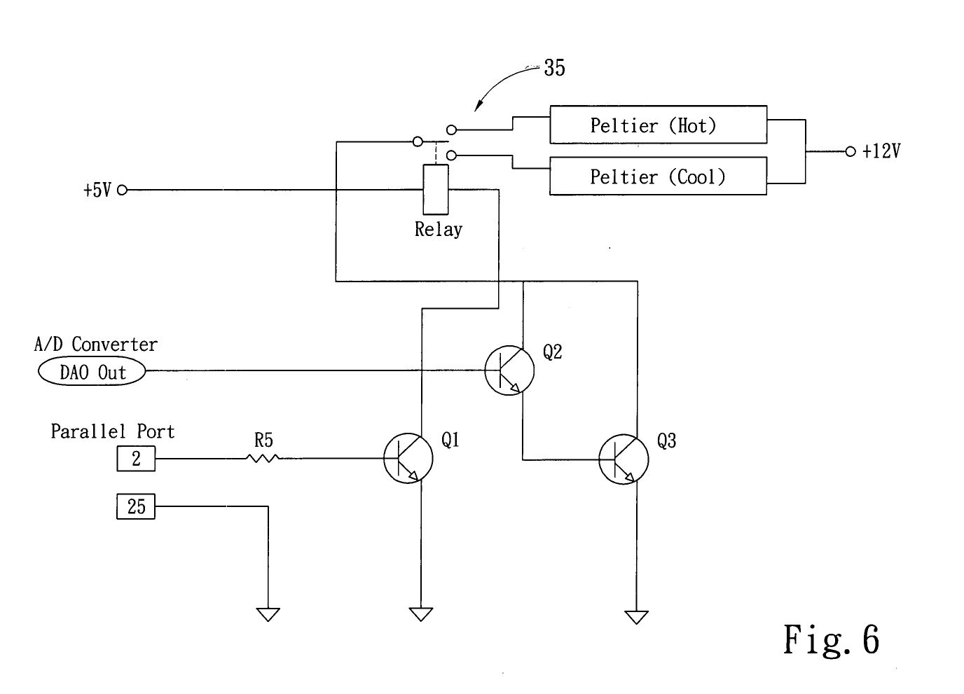

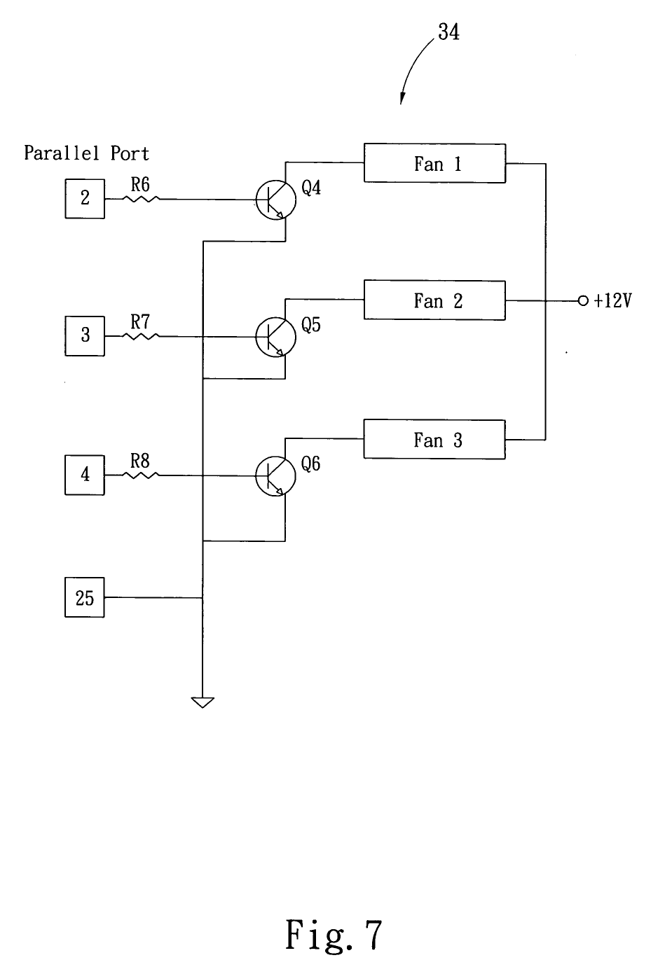

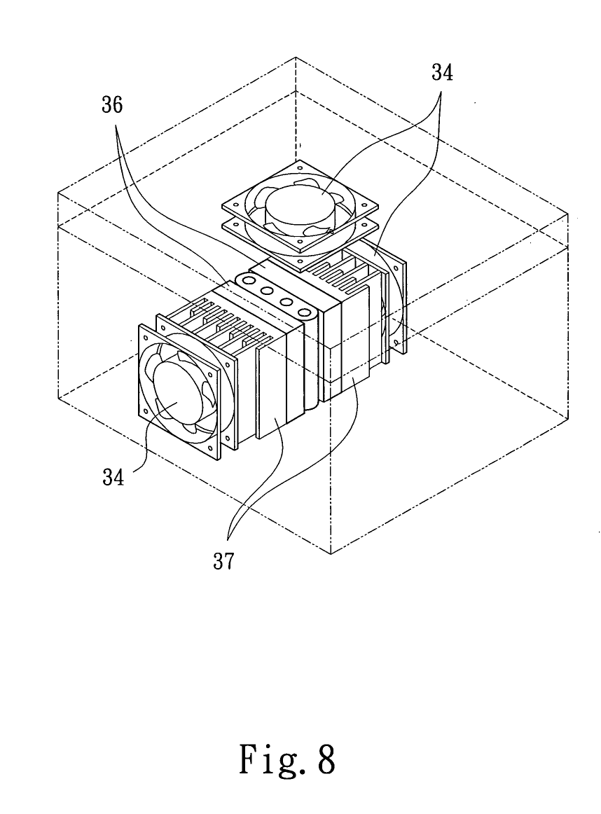

Referring in conjunction with FIG. 4-8, the temperature sensing system 10 can read an instant temperature (t) whose signal is then converted by an analog/digital converting circuit 21 and is transmitted to a microcomputer 30. In the microcomputer 30, the computer program therein will compare the signal with the temperature (To) preset by the temperature controlling system 10, and the result thus obtained is then transformed by a digital/analog converting circuit 33 and a voltage-current transformation circuit 35 to control the subsequent operation. This comparison result is divided into two parts:
1. When the temperature sensed is lower than the preset temperature (t<To), the microcomputer will deliver a signal to the thermoelectric semiconductor 36 to initiate the function of heating end of the thermoelectric semiconductor 36 and hence increases the temperature rapidly.
2. When the temperature sensed exceeds the preset temperature (t>To), the microcomputer 30 will deliver two signals through the same conducting route. One signal initiates the function of the cooling end of the thermoelectric semiconductor 36. At the same time, another signal will be transported by the microcomputer 30 through a parallel port to the fans 34 provided on both sides and above (as shown in FIG. 8) to activate fans 34. There are several heat dissipating pieces 37 interposed between said fan 34 and the thermoelectric semiconductor 36 in a manner that, after assembling, not only the external cool air can be drawn into the PCR tank by the fan 34, but also the hot air in the PCR tank can be exited by the top fan 34 such that the fan 34 can perform the heat dissipating effect. Consequently, the temperature in the PCR tank begins to lower by the combination of the thermoelectric semiconductor 36, the heat-dissipating piece 37 and the fan 34.

In an embodiment, PCR Positive Control Kit (E. coli tRNA₂^{fMet} genes) from We Gene Technologies® was used as the test sample. Concentrations and volume used of this test reagent is shown in Table 1. Firstly, temperatures in the course of the PCR were set as: denaturation (94° C. 15 sec), annealing (55° C., 15 sec), and extension (72° C. 30 sec). Then, a 30-cycles reaction was carried out. To the PCR product obtained after complete reaction was added in EtBr (Ethidium Bromide), the resulting mixture was then added in 2% agarose gel and an electrophoresis separation was performed for 30 minutes. As the electrophoresis was accomplished, the agarose gel was placed under ultraviolet light and photographed.

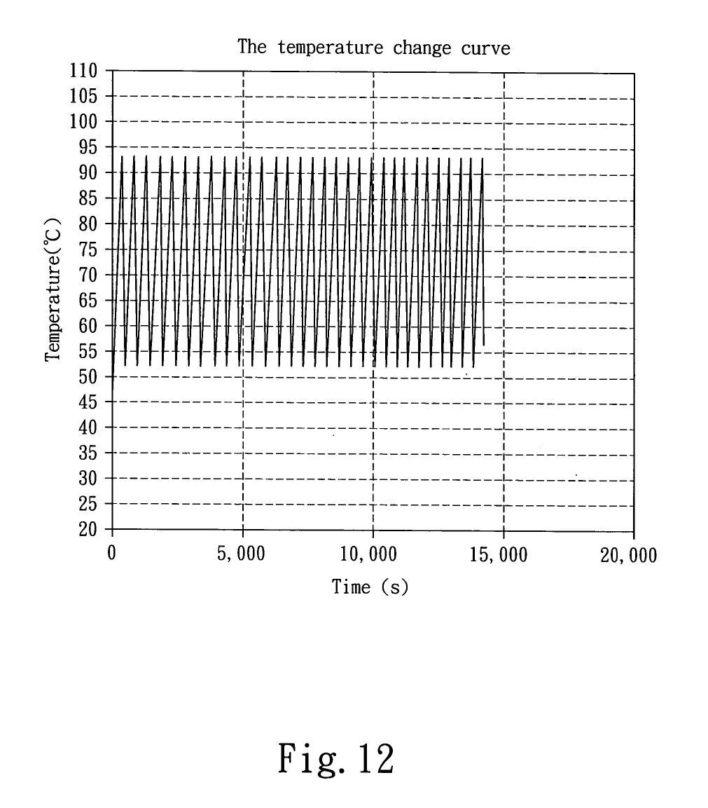

FIG. 12 shows the reaction course recorded within the 30-cycles reaction. The denaturation temperature was 94° C., the annealing temperature was 55° C. and extension temperature was 72° C. The whole reaction curve appeared cycling between 55° C.-94° C., and hold at 72° C. for a longer period.

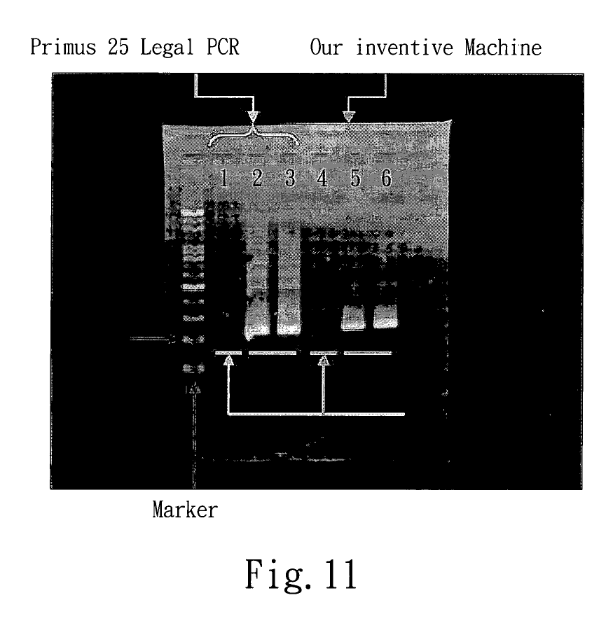

FIG. 11 shows photographs token on products obtained after =30-cycles PCR and separated by electrophoresis. Wherein, the fragment reproduced from E. Coli tRNA₂^{fMet} gene was 220 bp. Before PCR amplification, the concentration of this gene fragment was as low as could not be detected under ultraviolet irradiation. After amplified by the PCR machine according to the invention, this gene has a fragment size shown on the electrophoresis photograph just around the position of the marker of 220 bp.

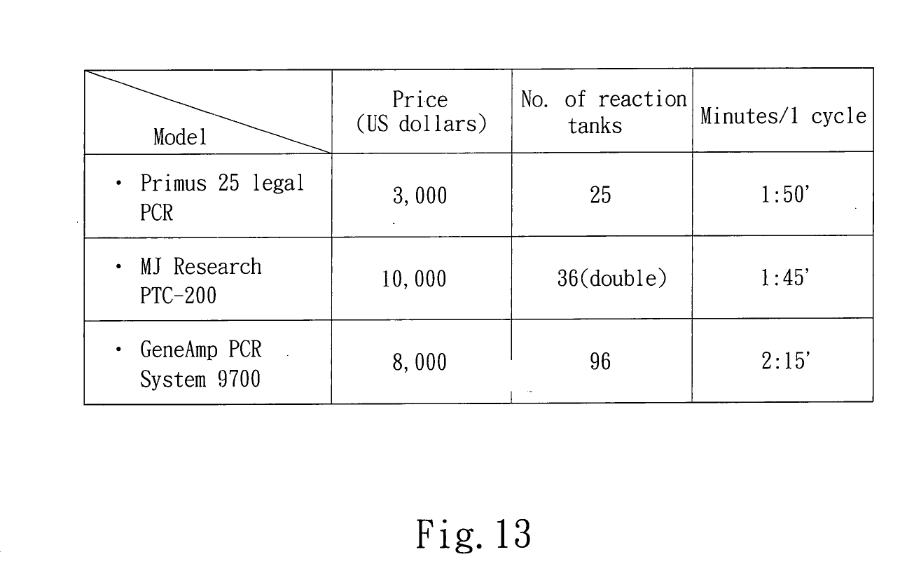

The PCR machine according to the invention are based on a design concept that can simplify steps involved in PCR compared with the conventional PCR machine (FIG. 13). Further, the novel process concept can reduce the production cost of a PCR machine to be 1/10- 1/15 that of the conventional PCR machine. This breakthrough not only can facilitate the popularization of the rapid medical diagnostic technology, but also exhibits a function as a portable machine.

The novel PCR machine according to the invention has several innovative features as follow:
1. A thermoelectric semiconductor heating method: A conventional PCR machine adopts generally a nickel-chrome wire electric resistor for heating, while the PCR machine employs thermoelectric semiconductor as an improved heating source, which offers two advantages as follow:
   (1) Its heating rate is faster than that of nickel-chrome wire electric resistor.
   (2) A uniform heat conduction can be provided at the contact surface between the thermoelectric semiconductor and the conductor such that every PCR heating tube can reach simultaneously at the preset temperature.
2. A combination of a thermoelectric semiconductor and fans is employed in the PCR machine according to the invention to offer a highly effective heat dissipation method. Most of the heat dissipation system used in the conventional PCR machines utilizes fans to lower the temperature with a temperature-lowering rate of about 0.6-0.9° C./s. In the PCR machine according to the invention, other than three fans, a thermoelectric semiconductor is provided to enhance cooling effect such that the temperature can be lowered more rapidly and PCR can be accomplished in a shorter time period.
3. The technique for measuring the reaction temperature is improved. The manner for measuring temperature in a conventional PCR machine comprises placing a temperature sensor on a conducting metal block, which will cause the temperature shown on the display panel being not equal to the actual temperature in the PCR heating tube. Since in the course of a PCR, whether a genetic material could be actually amplified is greatly associated with the precise control of the temperature, the temperature sensor is disposed in a movable nest-like container such that, when the PCR machine is to be used, the temperature sensor is placed in a solution that does not contain any genetic materials and has a volume equal to that of the reaction tube. This improvement can make the temperature in the PCR tube to be completely equal to that shown on the display.
4. The temperature controlling circuit is simplified. A conventional PCR machine uses a relatively complicated controlling circuit and a relatively large number of electronic elements. On the contrary, the PCR machine according to the invention addresses the design of electric circuit for controlling temperature by using an On-Off relay and transistor
5. A computerized controlling interface is used. Panels used in a conventional product are all LED-controlled panel. Although this can make operation with a single machine being possible, the display and setup in the course of reaction could not reflect real-time temperature change vs. time curve in the PCR. The innovative part of the PCR machine according to the invention resides on the ability of writing an easily operable interface so that, in addition to being able to carry out a simple operation using a mouse by the user, the record of temperature response curve at that time can be stored by the user for used in data analysis later.
6. It has a function of being portable. Due to the design of circuit and the improvement on the temperature increasing/lowering system, the volume of the whole PCR machine according to the invention can be reduced to the extent of being portable. This innovation can provide convenience of portability for the medical practitioner who has to go to remote mountain range or house call.
7. The heating or cooling efficiency around the PCR tube is increased. With respect to the heating and cooling in a conventional PCR machine, a variety of ways are used to control the temperature to vary in the range of 0-100° C. By way of example, the type of the PCR machine made by machine PCR manufacturer using Peltier heating employs exclusively Pletier heating, while regarding the heat dissipating manner, some machines use fan only, and others use Peltier, but they are all disposed below the sample tank. In general, there is a heat dissipation piece and a fan (none in some type of machine) provided below the Piltier. This underlying arrangement of heating and cooling means can provide reaction tube only 60% of heating efficiency. On the contrary, in the PCR machine according to the invention, Peltier elements are disposed around such that the effect of heating and cooling around the PCR tube can achieve a high efficiency of 95%. Furthermore, referring to FIG. 8, there is a fan provided at the upper opening of the reaction tank in the PCR machine according to the invention, which fan can draw off the internal heat, while fans at the left and right side can introduce the external air of lower temperature into the periphery of the reaction tank. The technique for controlling a plurality of Pletier is also a crucial factor. A good controlling technique can make the temperature around the reaction tube to reach the preset value, and can keep the temperature variation within ±0.1° C.

### INDUSTRIAL APPLICABILITY

As the application on clinical medicine, to a patient visiting the hospital for diagnosis due to pain in abdomen, the physician asks generally the patient for leaving a specimen for bacteria culture. After the specimen forming colony on a medium, the bacterial can be identified based on its morphological characteristics and biochemical reaction. Albeit this traditional process is accurate, it must take usually 1 to 7 days to accomplish the identification. Further, there might be diagnostic error due to the complicated chemical reaction and/or the experience factor of the technician. Moreover, in case of infection sources such as virus, the cultivation method may not be effective at all, such that the physician might not achieve an accurate diagnosis but can only prescribe some antibiotic drugs. As an example of this circumstance, mention can be made of the enterovirus epidemic occurred in 1998 (information provided by Enterovirus Section, Virological Disease Group, Disease Control Center, Department of Health, Executive Yuan, ROC), the physician in a general clinic can postulate the possibility of enterovirus infection based on superficial symptoms, whereas for further identification of the type of the enterovirus infection source, a PCR molecular biological examination and the like must be performed in the examination laboratory of a hospital. In this situation, time spent from the arrival of the specimen to the outcome of the identification exceeds the latent period of three days. Thus, in case of the infection of the serious enterovirus type 71, its mortality will be increased owing to the delay. Therefore, if the PCR equipment can be distributed to every clinic, the physician can identify rapidly and accurately the cause of the lesion, e.g., the pathogenic bacteria that infects the patient

As on the improvement for the agriculture, Bacillus thuringiensis is a bacterium that can damage to insects belonging to Lepidoptera, Coleoptera, and Diptera. When the insect uptakes the Bacillus thuringiensis, the insecticidal crystalline protein contained in this bacterium can cause the imbalance of the osmosis pressure in the intestinal tract of that insect and then the intestinal cell is broken to collapse the intestinal tract and death of the insect (Payne et al. 1995). As the technology advances, researchers have clone the insecticidal gene of Bacillus thuringiensis into some plants such as, for example, tomato, potato, cabbage and the like by means of genetic engineering technology such as PCR technique and recombination DNA technique. As the result, these plants become transgenic crop having insecticidal activity. Bacillus thuringiensis can only causes damage to particular insects and harmless to human being.

As for forensic diagnosis, PCR technique can be a strong and advantageous tool for the identification of a crime. In case the criminal leaves a hair or even a drop of blood at the venue, these specimen can facilitate the identity of the criminal by means of PCR technique. By virtue of this criminal identification technique, there will be no room for verbal defense left to the criminal.

Those embodiments of applications mentioned above are only a small part in vast applications of the novel polymerase chain reactor according to the invention. The scope of the application of PCR technique is actually very broad that cannot list exhaustively. Accordingly, the polymerase chain reactor according to the invention has really an improved structure, is innovative in term of the overall spatial configuration and exhibits an obvious enhancement on effectiveness compared with the conventional polymerase chain reactor.

While the invention has been described with reference to preferred embodiments thereof, it should be understood that the scope of the invention is not limited by these embodiments. Many changes and modifications in the above described embodiment of the invention can, of course, be carried out without departing from the scope thereof. Accordingly, to promote the progress in science and the useful arts, the invention is disclosed and is intended to be limited only by the scope of appended claims.

TABLE 1

| | Ingredients | Volume added(μL) |
|---|---|---|
| 1. | Primer F (100 ng/μL) | 0.5 |
| 2. | Primer R (100 ng/μL) | 0.5 |
| 3. | dNTPs Mixture (2.5 mM/each) | 0.8 |
| 4. | 10X Buffer (with 15 mM MgCl₂) | 1 |
| 5. | ddH₂O (PCR grade) | 6.1 |
| 6. | Taq DNA Polymerase | 0.1 |
| | (pH8.0, PCR grade) | |
| 7. | Template (E.coli JM 109 genomic | 1 |
| | DNA, 10³ copies/μL) | |
| | Total | 10 |

1. A polymerase chain reactor, comprises essentially a temperature measuring system; a temperature controlling system; and a human machine interface, characterized in that
said temperature measuring system comprises a temperature sensor to measure the temperature in the reaction tube in the course of the reaction; the temperature thus measured is converted into an electric signal by an analog/digital converter and the electric signal is then transmitted to a microcomputer where said electric signal is converted into temperature through an electric signal-temperature transformation program and is displayed on a operational interface;
that said temperature controlling system is consisted of a computer-controlled thermoelectric semiconductor, two heat dissipating piece and three groups of fan that are provided on three sides of the polymerase chain reaction (PCR) tank, i.e., said thermoelectric semiconductor is interposed between a heat dissipating piece and a fan, while another fan is disposed above said thermoelectric semiconductor, such that said microcomputer can deliver a signal via parallel port to fans provided on both sides of and above said thermoelectric semiconductor to activate said fans, thereby fans on both sides can draw the external cool air into the PCR tank, while the upper fan can expel the hot air in the PCR tank to act the function of heat dissipation and hence, by means of the combination of said thermoelectric semiconductor, said heat dissipation piece and said fans, the temperature in the PCR tank begins to lower; wherein, after setting the temperature program for the polymerase chain reaction, by means of said microcomputer, said temperature measuring system will be commanded to measure the real time reaction temperature, said temperature controlling system will control and adjust instantly the reaction temperature based on the reaction temperature program set in the microcomputer, and the temperature will be then displayed on the screen of the operation interface so that the operator can set based on the required operation procedure, and can select an automatic administration procedure; wherein the result of said operation and course can be stored for inspection and analysis later; and that the temperature of the air inside said polymerase chain reactor can be lowered by the combination of said thermoelectric semiconductor said heat dissipation pieces and said fans.

2. A polymerase chain reactors as in claim 1, wherein said temperature sensor in said temperature measuring system is a microthermistor.

3. A polymerase chain reactors as in claim 1, wherein said temperature sensor in said temperature measuring system is a heat resistant, corrosion resistant element, and is disposed in the reagent contained in a reaction tube during the polymerase chain reaction (PCR).

4. A polymerase chain reactors as in claim 1, wherein said microcomputer in said temperature controlling system is used to control said fans, and is provided in parallel with an analog/digital converting circuit and a voltage-current transformation circuit so as to control the increase or lowering of the temperature of said thermoelectric semiconductor.

5. A polymerase chain reactors as in claim 4, wherein as said microcomputer in said temperature controlling system detects a temperature in the reaction tube lower than said preset temperature, said microcomputer will delivers an electric signal that, after being converted by said analog/digital converting circuit and transformed by said voltage-currrent transformer, can activate the function of heating end of said thermoelectric semiconductor as being the heat supply end.

6. A polymerase chain reactors as in claim 4, wherein as said microcomputer in said temperature controlling system detects a temperature in the reaction tube higher than the preset temperature, said microcomputer will delivers two signals, one signal used to control said fans directly, and other signal, after being converted by said analog/digital converting circuit and transformed by said voltage-current transformer, used to activate the cooling end of said thermoelectric semiconductor and to dissipate heat in conjunction with said fans.

7. A polymerase chain reactors as in claim 1, wherein as said human machine interface comprises a display to display and store the course and result of the reaction.

8. A polymerase chain reactors as in claim 1, wherein as said human machine interface can be used further in conjunction with a personal computer, and the user can change reaction conditions at any time as desired.
